(19) United States
(12) Patent Application Publication
(10) Pub. No.: US 2003/0187437 A1
(43) Pub. Date: Oct. 2, 2003

(54) Serrated spinal hook

(75) Inventor: Glen M. Ginsburg, Omaha, NE (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY ACROMED, INC.

(21) Appl. No.: 10/113,904

(22) Filed: Mar. 29, 2002

(51) Int. Cl. A61B 17/58

(52) U.S. Cl. 606/61

(57) ABSTRACT

A serrated pedicle hook for use in a spinal fixation system is provided. The pedicle hook includes a body portion that is adapted to engage the fixation element of a spinal fixation system, and a hook portion that is adapted to be positioned on and/or around the pedicle bone of a vertebra. The hook portion includes at least one serration formed on the bone-contacting surface. The serrated spinal hook according to the present invention is particularly useful for maintaining the desired position of a pedicle hook both during and after the implantation of a spinal fixation system.

### FIELD OF THE INVENTION

The present invention relates to spinal fixation devices, and in particular to a pedicle hook that is effective to engage pedicle bone.

### BACKGROUND OF THE INVENTION

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. The fixation rods can have a predetermined contour that has been designed according to the properties of the target implantation site, and once installed, the instrument holds the vertebrae in a desired spatial relationship, either until desired healing or spinal fusion has taken place, or for some longer period of time.

Spinal fixation devices can be anchored to specific portions of the vertebra. Since each vertebra varies in shape and size, a variety of anchoring devices have been developed to facilitate engagement of a particular portion of the bone. Pedicle hook assemblies, for example, have a shape and size that is configured to engage pedicle bone. Such hooks typically include a blade portion that is adapted to conform to the inner (ventral) surface of the inferior thoracic facet engaging the pedicle bone, and a shank portion having a rod-receiving element, usually in the form of a U-shaped slot formed in the shank. A cap, or similar type of locking mechanism, is used to lock the fixation rod to the hook assembly.

In use, the ends of the fixation rod are each loosely secured to a hook assembly. The blade portion of each hook is then moved into engagement with the pedicle bone of the vertebrae, and once properly positioned, the fixation rod is locked in place by tightening a cap or similar type of locking mechanism to securely interconnect each hook and the fixation rod.

While current spinal fixation systems have proven effective, difficulties have been encountered in mounting various fixation devices on the rods, and maintaining them in a desired position and orientation with respect to the rod. In particular, it can be difficult to maintain the position of the hook relative to the spinal column before the assembly is locked, particularly when the fixation rod is being inserted into the rod-receiving element of the hook assembly. Thus, there presently exists a need for a hook that is effective to securely engage pedicle bone to prevent movement of the hook assembly with respect to the bone during surgical placement and/or after installation.

### SUMMARY OF THE INVENTION

The present invention provides a serrated spinal hook for maintaining the position of a pedicle hook both during and after the implantation of a spinal fixation system. In general, the pedicle hook of the present invention has two portions, a body portion that engages the fixation element of the spinal fixation system and a hook portion that is placed on and/or around the pedicle bone of a vertebra. The hook portion has at least one serration, which is formed on the surface that ultimately contacts the pedicle bone. This serration allows the pedicle hook to be inserted in one direction, while simultaneously hindering removal in a second, opposite direction.

### BRIEF DESCRIPTION OF THE DRAWINGS

The invention will be more fully understood from the following detailed description taken in conjunction with the accompanying drawings, in which:

FIGS. 1A-1C are perspective view illustrations of a spinal fixation system having a pedicle hook according to the present invention;

FIG. 2 is a perspective view illustration of a pedicle hook according to an exemplary embodiment of the present invention;

FIG. 3 is a side view illustration of the pedicle hook of FIG. 2;

FIG. 4 is an enlarged view of the of the lower blade portion of the pedicle hook of FIG. 3;

FIG. 5 is an enlarged view of another embodiment of the lower blade portion of the pedicle hook of the present invention.

### DETAILED DESCRIPTION OF THE INVENTION

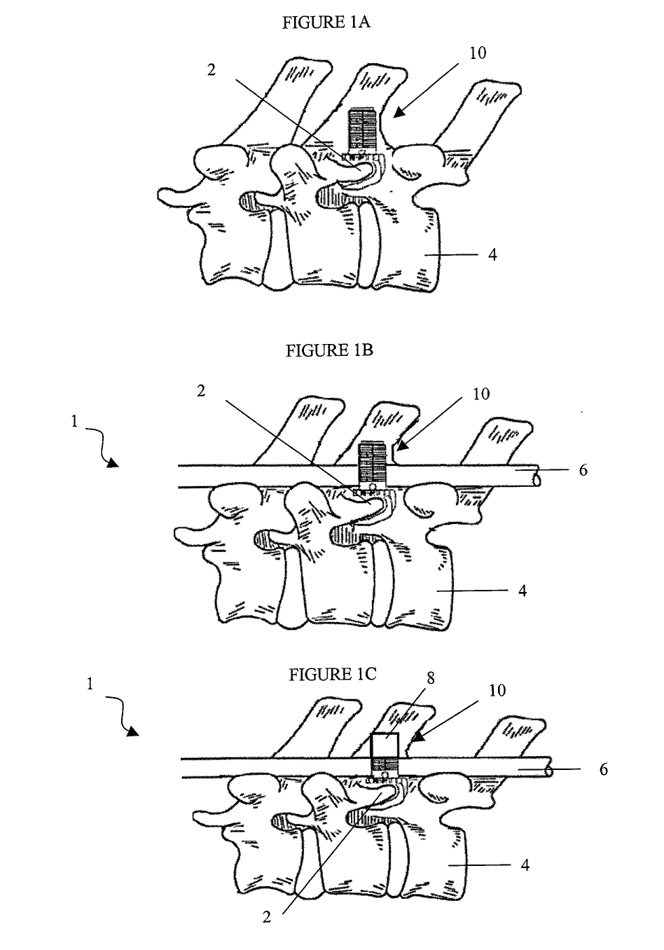

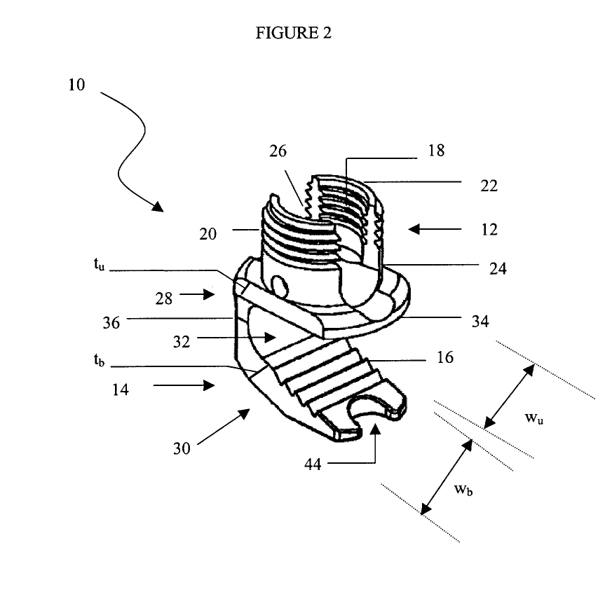

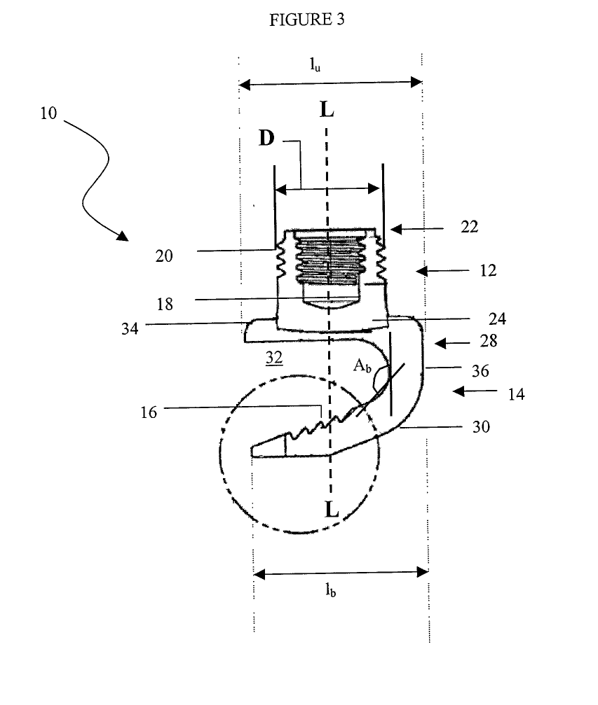

The present invention provides a pedicle hook 10 that is effective to engage a pedicle bone 2 of a vertebra 4. In general, the pedicle hook 10 of the present invention is particularly useful in spinal fixation systems 1 that are designed to hold vertebrae 4 in a desired spatial relationship. Spinal fixation systems 1 typically include one or more hook assemblies that are adapted to engage a patient's vertebrae 4, a fixation element that spans two or more vertebrae 4, and a locking mechanism that is effective to securely interconnect the hook assembly and the fixation element. FIGS. 1A-1C illustrate a spinal fixation system 1 that includes the pedicle hook 10 of the present invention, a relatively rigid fixation rod 6 and a locking mechanism that comprises an end cap 8. As shown in FIGS. 2 and 3, the pedicle hook 10 of the present invention includes an elongate body portion 12 and a hook member 14 having an overall size and geometry that allow the pedicle hook 10 to be efficiently installed within a patient and to remain securely positioned on a pedicle bone 2. According to the present invention, the hook member 14 includes at least one serration 16, which is formed on the bone-contacting surface of the hook member 14. The at least one serration 16 is advantageous, because the serration(s) 16 prevents the pedicle hook 10 from moving while the spinal fixation system is surgically placed within a patient. The serration(s) 16 also prevents the pedicle hook 10 from moving and/or shifting after the spinal fixation system 1 has been implanted within a patient.

The elongate body portion 12 of the pedicle hook 10 can have virtually any shape, but preferably, the elongate body portion 12 has a shape that is effective to receive a fixation element, such as the fixation rod 6 shown in FIGS. 1B and 1C. Preferable shapes for the elongate body portion 12, i.e., shapes that are effective to receive a fixation element, are known in the art. In an exemplary embodiment of the pedicle hook 10, shown in FIGS. 2 and 3, the elongate body portion 12 has a generally cylindrical shape.

As shown in FIGS. 2 and 3, the elongate body portion 12 has an inner surface 18 and an opposed outer surface 20. The distance between any two points on the outer surface 20, which are connected by a straight line passing through the center of the elongate body portion 12, defines a diameter D of the elongate body portion 12, as shown in FIG. 3. This diameter D can vary, but preferably, the diameter D of the elongate body portion 12 is between about 2 and 24 mm.

In one embodiment of the present invention, at least a portion of the outer surface 20 of the elongate body portion 12 is threaded. Optionally, the inner surface 18 can also contain at least a portion that is threaded. The size and geometry of the threaded portion(s) can vary, but preferably, at least the threaded portion of the outer surface 20 is effective to mate with and/or to engage a locking element of a spinal fixation system 1, such as for example, the end cap 8 illustrated in FIG. 1C.

Referring to FIG. 3, the elongate body portion 12 also has a first end 22, a second end 24, and a longitudinal axis L that extends between the two ends. The distance between the first end 22 and the second end 24, measured along the longitudinal axis L, defines the length of the elongate body portion 12. This length can vary, but preferably, the length of the elongate body portion 12 is determined and adjusted according to the dimensions of the target site of implantation. More preferably, the elongate body portion 12 has a length between about 3 and 50 mm.

As shown in FIGS. 2 and 3, the first end 22 of the elongate body portion 12 can have virtually any shape, but preferably the first end 22 contains a recess 26, which extends through opposite walls that define the elongate body portion 12. This recess 26 can have virtually any size and geometry, but preferably, the recess 26 has a size and shape that are effective to receive the fixation element of a spinal fixation system 1, such as for example, the fixation rod 6 illustrated in FIGS. 1B and 1C. In an exemplary embodiment of the present invention shown in FIGS. 2 and 3, the recess 26 has a U-shape that allows a fixation rod 6 to sit within the elongate body portion 12.

Referring to FIGS. 2 and 3, the pedicle hook 10 of the present invention also includes a hook member 14, which extends from the elongate body portion 12. The hook member 14 can be integrally formed with the elongate body portion 12, or alternatively, the hook member 14 and the elongate body portion 12 can comprise separate elements that have been mated together to form a pedicle hook 10 according to the present invention. The elongate body portion 12 and the hook member 14 can be coupled together using a variety of mating techniques known to those having ordinary skill in the art. By way of non-limiting example, the hook member 14 can be welded, ultrasonically bonded, adhesively attached or mechanically mated to the elongate body portion 12.

The hook member 14 of the present invention can have virtually any shape. In the -exemplary embodiment illustrated in FIGS. 2 and 3, the hook member 14 has an upper portion 28 and a lower blade portion 30 that are shaped to engage pedicle bone 2. While these elements are described herein as portions, one of ordinary skill in the art will appreciate that the upper and lower portions 28, 30 respectively, can be integrally formed, or alternatively, they can comprise two separate elements that have been mated together using any of the mating techniques known in the art.

The upper and lower portions 28, 30 of the hook member 14 can have virtually any shape, but preferably, these portions are shaped to form an opening or recess 32 that allows the pedicle hook 10 of the present invention to receive and engage pedicle bone 2. This recess 32 can have virtually any size and geometry, but preferably, the size and shape of recess 32 are determined and adjusted according to the dimensions of the target site of implantation, i.e. the pedicle bone 2. In the exemplary embodiment shown in FIGS. 2 and 3, the hook member 14 is generally C-shaped.

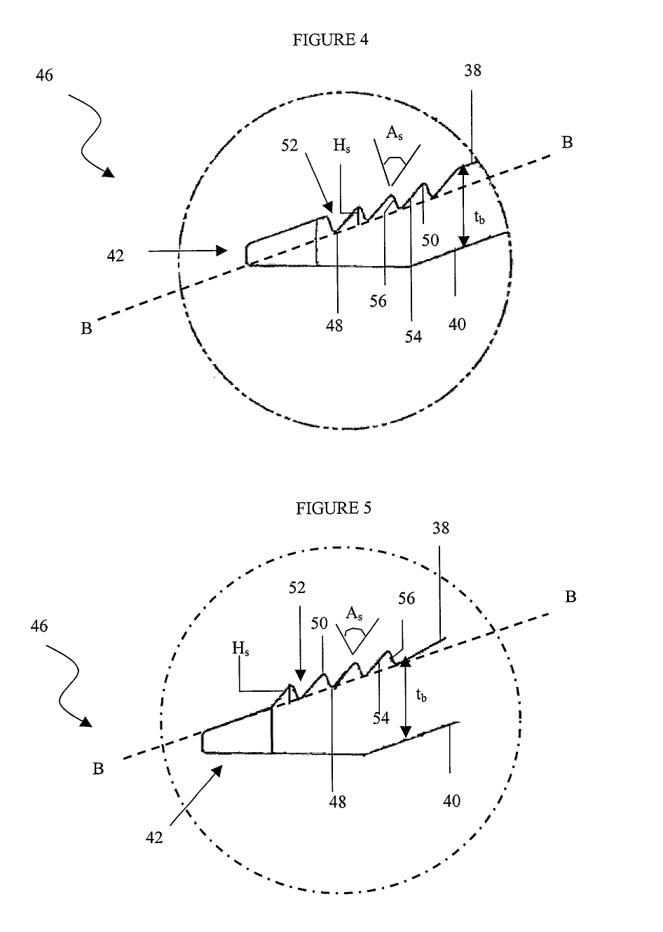

Referring to FIG. 2, the upper portion 28 and the lower blade portion 30 each have a thickness, labeled as tᵤ and t_{b}. The upper and lower portions 28, 30 can have virtually any thickness. For example, the thickness of the upper portion tᵤ can be smaller than, greater than, or substantially equal to the thickness of the lower blade portion t_{b}. Furthermore, the thickness of each portion tᵤ, t_{b} can be uniform throughout the upper or lower blade portions 28, 30 or alternatively, the thickness tᵤ, t_{b} can vary within each portion. In an exemplary embodiment illustrated in FIGS. 2 and 3, the thickness of the lower blade portion t_{b} is tapered so as to decrease toward the distal tip 42 (FIGS. 4 and 5) of the lower blade portion 30. Preferably, the upper portion 28 has a thickness tᵤ that is sufficient to provide structural integrity to the pedicle hook 10 and to provide rigid support for the lower blade portion 30, while the lower blade portion 30 has a thickness t_{b} that is sufficient to provide structural integrity to the pedicle hook 10 such that the lower blade portion 30 can effectively and securely engage pedicle bone 2 without breaking or other damage. More preferably, the thickness of each portion, tᵤ, t_{b} is in the range of about 1 to 30 mm.

As shown in FIG. 2, the upper portion 28 and the lower portion 30 of the pedicle hook 10 each also have a width, labeled as wᵤ and w_{b} in FIG. 2. The upper and lower portions 28, 30 can have virtually any width. For example, the width of the upper portion wᵤ can be smaller than, greater than, or substantially equal to the width of the lower blade portion w_{b}. Additionally, the width of the upper and lower portions wᵤ, w_{b} can be uniform throughout each portion, or alternatively, the width within each portion wᵤ, w_{b} can vary. Preferably, the width of the upper portion wᵤ is substantially equal to, or greater than, the diameter D across the elongate body portion 12, while the width of the lower blade portion w_{b} is sufficient to allow the lower blade portion 30 to securely engage pedicle bone 2. More preferably, the width of each portion wᵤ, w_{b} is in the range of about 2 to 24 mm.

The upper lower portions 28, 30 each also have a length, labeled as lᵤ and l_{b} in FIG. 3. The upper portion 28 can have virtually any length, but preferably, the length lᵤ of the upper portion 28 is substantially equal to, or greater than, the diameter D across the elongate body portion 12. More preferably, the upper portion 28 has a length lᵤ that is greater than the diameter D of the elongate body portion 12 in at least the direction in which the lower blade portion 30 extends from the upper portion 28, thereby allowing the pedicle hook 10 to grip and/or engage a larger area of pedicle bone 2. The lower blade portion 30 can have virtually any length, but preferably the length of the lower blade portion l_{b} is determined and adjusted according to the dimensions of the intended implantation site, thereby allowing the lower blade portion 30 to securely engage the pedicle bone 2. More preferably, the length of each portion lᵤ, l_{b} is in the range of about 4 to 30 mm.

In the exemplary embodiment shown in FIGS. 2 and 3, the upper portion 28 comprises two components: a transverse component 34 and a distally extending component 36, which extends from and is integral with the transverse component 34. While the transverse component 34 and distally extending component 36 are described herein as components, or portions, of the hook member 14, one of ordinary skill in the art will appreciate that these components can also comprise separate elements. In this alternative embodiment, the transverse component 34 and distally extending component 36 can be mated together using any of the various mating techniques known to those having ordinary skill in the art. The distally extending component 36 can extend from the transverse component 34 in virtually any orientation, and thus, the upper portion 28 of the pedicle hook 10 can have virtually any shape. In an exemplary embodiment of the pedicle hook 10 illustrated in FIGS. 2 and 3, the distally extending component 36 extends from the transverse component 34 in a direction that is substantially parallel to the longitudinal axis L of the elongate body portion 12 and substantially perpendicular to the transverse component 34.

As seen in FIGS. 2 and 3, the lower blade portion 30 extends from the upper portion 28 of the hook member 14. The lower blade portion 30 and the upper portion 28 can be integrally formed, or alternatively, the lower blade portion 30 can be mated to the distally extending component 34 by any of the mating techniques known to those having ordinary skill in the art. Furthermore, the lower blade portion 30 can extend from the upper portion 28 in virtually any configuration. Preferably, the lower blade portion 30 extends at angle with respect to the upper portion 28. The angle at which the lower blade portion 14 extends, labeled as A_{b} in FIG. 3, can vary, but preferably, the lower blade portion 30 is angled to extend across the longitudinal axis L of the elongate body portion 12. More preferably, the lower blade portion 30 extends at an obtuse angle A_{b}. Even more preferably, the angle at which the lower blade portion A_{b} extends is between about 20 and 150 degrees.

As seen in FIGS. 4 and 5, the lower blade portion 30 has an inner, bone-contacting surface 38 and an outer surface 40 that define the thickness t_{b} of the lower blade portion 30, as shown in FIGS. 4 and 5. As described above, this thickness t_{b} can vary but preferably the thickness of the lower blade portion t_{b} is tapered such that it decreases toward the distal tip 42. The distal tip 42 of the lower blade portion 30 can have virtually any shape. As shown in FIG. 2, the distal tip 42 can optionally contain an indentation or recess 44 formed therein. Preferably, this recess 44 is adapted to provide clearance around the pedicle bone 2, thereby allowing the pedicle hook 10 to engage the pedicle bone 2 more effectively.

The lower blade portion 30 also includes a distal portion 46, which extends from the longitudinal axis L of the elongate body portion 12 to a distal tip 42. This distal portion 46 can extend from the longitudinal axis L at virtually any angle, but preferably, this portion 46 extends in an orientation that is substantially perpendicular to the longitudinal axis L. The distal portion 46 of the lower blade portion 30 can have virtually any shape, but preferably, the distal portion 40 has a wedge-like shape, in which the thickness of the lower blade portion t_{b} decreases toward the distal tip 42, as illustrated in FIGS. 4 and 5.

The lower blade portion 30 also includes at least one serration 16 formed on the inner bone-contacting surface 38 of the lower blade portion 30. The lower blade portion 30 can include virtually any number of serration(s) 16, but preferably, the number of serration(s) 16 is between 1 and about 10. Each serration 16 can have virtually any shape, but preferably, the serration(s) 16 has a ridge-like shape that contains a base 48 and a peak 50 that are angled to form a recess 52 that is defined by a first side 54 and a second side 56, as shown in FIGS. 4 and 5. The recess 52 formed in each serration 16 can be formed as an indentation that has been cut into the inner, bone-contacting surface 38 of the lower blade portion 30, as shown in FIG. 4, or alternatively, the recess 52 can be formed as a protrusion that extends above the inner surface 38, as shown in FIG. 5. The height of each serration 16, labeled as Hₛ in FIGS. 4 and 5, is defined as the distance between each peak 50 and a straight line connecting each base 48, i.e. the baseline, labeled as line B-B in FIGS. 4 and 5. The height of each serration Hₛ can vary, but preferably, the height of each serration Hₛ is in the range of about 0.1 to 5 mm.

According to the present invention, the first and second sides 54, 56 of each serration 16 can be aligned in virtually any configuration, but preferably, the first and second sides 54, 56 are angled with respect to each other, thereby forming the angle of each serration, labeled as Aₛ in FIGS. 4 and 5. The angle between the first and second sides Aₛ can vary but preferably, the angle Aₛ is sufficient to allow insertion in a first direction, while simultaneously impeding and/or preventing removal in a second, opposite direction. More preferably, the angle of each serration Aₛ is in the range of about 10 to 90 degrees.

The pedicle hook 10 of the present invention is adapted for use in a spinal fixation system 1. By way of non-limiting example, a spinal fixation system 1 according to the present invention can include two pedicle hooks 10, a fixation rod 6, and a locking mechanism, such as for example, the end cap 8 illustrated in FIG. 1C. The first end 22 of the elongate body 12 of the pedicle hook 10 is effective to receive the fixation rod 6, while the hook member 14 of the pedicle hook 10 is adapted to engage pedicle bone 2. The locking mechanism 8 is effective to engage the elongate body portion 12 of the pedicle hook 10 and to tightly secure the fixation rod 6 within the first end 22 the pedicle hook 10. In use, the pedicle hooks 10 are first placed loosely on the target pedicle bone 2, while the fixation rod 6 is seated within the first end 22 of the elongate body portion 12, as shown in FIGS. 1A and 1B. Once the fixation rod 6 has been placed within the first end 22 of the elongate body 12, the locking mechanism 8 is then placed onto the first end 22 of the elongate body portion 12 and tightened to securely interconnect the fixation rod 6 and the pedicle hooks 10, as illustrated in FIG. 1C.

The pedicle hook 10 of the present invention can be constructed from virtually any material that is biologically compatible and suitable for medical applications. One preferred material from which the pedicle hook 10 can be made is stainless steel. Other materials from which the pedicle hook 10 can be constructed include titanium.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A pedicle hook, comprising:
an elongate body portion having a first end, a second end, and a longitudinal axis extending therebetween;
a hook member having an upper portion mated to the second end of the body and extending distally from the body in a direction substantially parallel to the longitudinal axis of the body, and a lower blade portion extending at an angle with respect to the upper portion such that the lower blade portion extends across the longitudinal axis of the body, the lower blade portion including an inner surface and an outer surface, wherein the inner surface has at least one serration formed thereon that is adapted to engage pedicle bone.

2. The pedicle hook of claim 1, wherein the body portion has a generally cylindrical shape and includes at least one recess formed in the first end, the recess being adapted to receive a fixation rod.

3. The pedicle hook of claim 2, wherein the upper portion is substantially L-shaped and includes a transverse component mated to the second end of the body and a distally extending component mated to the lower blade portion of the hook member.

4. The pedicle hook of claim 3, wherein the distally extending component of the upper portion of the hook member is positioned at a distance from the longitudinal axis of the elongate body such that that the distally extending component is offset with respect to the longitudinal axis.

5. The pedicle hook of claim 1, wherein the lower blade portion extends at an angle with respect to the upper portion between about 20 and 150 degrees.

6. The pedicle hook of claim 1, wherein the lower blade portion of the hook member has a thickness extending between the inner and outer surfaces, and a width extending from a first side of the inner and outer surfaces to a second, opposed side of the inner and outer surfaces.

7. The pedicle hook of claim 6, wherein the lower blade portion of the hook member includes a distal tip, and the thickness of the lower blade portion decreases toward the distal tip.

8. The pedicle hook of claim 7, wherein the distal tip includes an indented portion formed therein.

9. The pedicle hook of claim 6, wherein the thickness extending between the inner and outer surfaces is in the range of about 1 and 30 mm.

10. The pedicle hook of claim 6, wherein the width extending between the first and second sides of the inner and outer surfaces is in the range of about 2 to 24 mm.

11. The pedicle hook of claim 6, wherein each of the at least one serrations extends between the first and second sides of the inner surface and forms a ridge-like member.

12. The pedicle hook of claim 11, wherein the at least one serration is angled to allow the pedicle hook to extend around the pedicle bone when moved in a first direction, while engaging the pedicle bone to secure the pedicle hook to the bone when moved in a second, opposite direction.

13. The pedicle hook of claim 1, wherein the elongate body portion includes an outer surface extending between the first and second ends thereof, the outer surface having at least one threaded portion formed thereon.

14. The pedicle hook of claim 2, wherein the elongate body portion includes at least one threaded portion formed on at least one of the recess and an outer surface of the body.
